(19) United States
(12) Patent Application Publication
(10) Pub. No.: US 2021/0038280 A1
(43) Pub. Date: Feb. 11, 2021

(54) ELECTROSURGICAL GENERATOR FOR OPTIMIZING POWER OUTPUT

(71) Applicant: John Pikramenos, Rocky Point, NY (US)

(72) Inventor: John Pikramenos, Rocky Point, NY (US)

(21) Appl. No.: 16/535,629

(22) Filed: Aug. 8, 2019

(51) Int. Cl. A61B 18/12; H04L 29/08; A61B 90/98; A61B 34/00

(52) U.S. Cl. CPC A61B 18/1206; H04L 67/12; H04B 5/0062; A61B 34/25; A61B 90/98

(57) ABSTRACT

An electrosurgical generator is presented for controlling a surgical instrument. The electrosurgical generator includes a controller programmed to generate a first carrier wave signal and a second carrier wave signal based on an algorithm employing real-time current values of tissue at a tissue site to determine tissue impedance at a distal end of the surgical instrument, the first carrier wave signal having a first oscillating waveform and the second carrier wave signal having a second oscillating waveform, a multistage variable gain amplifier for amplifying the first and second oscillating waveforms to generate a first output signal for a first mode of operation and a second output signal for a second mode of operation, and an electrosurgical connector for transmitting the first and second output signals. The controller concurrently runs the first and second oscillating waveforms while switching between the first mode of operation and the second mode of operation.

### BACKGROUND

The present invention relates generally to electrosurgical generators, and more specifically, to electrosurgical generators for optimizing power output.

Electrosurgery involves the application of high-frequency electric current to cut or modify biological tissue during an electrosurgical procedure. Electrosurgery is performed using an electrosurgical generator, an active electrode, and a return electrode. The electrosurgical generator (also referred to as a power supply or waveform generator) generates an alternating current (AC), which is applied to a patient's tissue through the active electrode and is returned to the electrosurgical generator through the return electrode. The alternating current typically has a frequency above 100 kilohertz (kHz) to avoid muscle and/or nerve stimulation.

During electrosurgery, the AC generated by the electrosurgical generator is conducted through tissue disposed between the active and return electrodes. The tissue's impedance converts the electrical energy (also referred to as electrosurgical energy) associated with the AC into heat, which causes the tissue temperature to rise. The electrosurgical generator controls the heating of the tissue by controlling the electric power (i.e., electrical energy per time) provided to the tissue. Although many other variables affect the total heating of the tissue, increased current density usually leads to increased heating. The electrosurgical energy is typically used for cutting, dissecting, ablating, coagulating, and/or sealing tissue.

The two basic types of electrosurgery employed are monopolar and bipolar electrosurgery. Both of these types of electrosurgery use an active electrode and a return electrode. In bipolar electrosurgery, the surgical instrument includes an active electrode and a return electrode on the same instrument or in very close proximity to one another, which cause current to flow through a small amount of tissue. In monopolar electrosurgery, the return electrode is located elsewhere on the patient's body and is typically not a part of the electrosurgical instrument itself. In monopolar electrosurgery, the return electrode is part of a device typically referred to as a return pad.

Electrosurgical generators make use of voltage and current sensors to measure quantities, such as power and tissue impedance, for controlling the output of the electrosurgical generator to achieve a desired clinical effect. The voltage and current sensors are often located inside the electrosurgical generators to save costs associated with incorporating sensors into the surgical instruments.

### SUMMARY

In accordance with an embodiment, an electrosurgical generator for controlling a surgical instrument is provided. The electrosurgical generator includes a controller programmed to generate a first carrier wave signal and a second carrier wave signal based on an algorithm executed on a processor employing real-time current values of tissue at a tissue site to determine tissue impedance at a distal end of the surgical instrument, the first carrier wave signal having a first oscillating waveform and the second carrier wave signal having a second oscillating waveform, a multistage variable gain amplifier for amplifying the first and second oscillating waveforms to generate a first output signal for a first mode of operation and a second output signal for a second mode of operation; and an electrosurgical connector for transmitting the first and second output signals to one or more electrodes on the surgical instrument, wherein the controller concurrently runs the first and second oscillating waveforms while switching between the first mode of operation and the second mode of operation.

In accordance with another embodiment, an electrosurgical generator for controlling a surgical instrument is provided. The electrosurgical generator includes a controller programmed to generate a carrier wave signal based on an algorithm executed on a processor employing real-time current values of tissue at a tissue site to measure at least one of phase angle and reactance of the tissue, the carrier wave signal having an oscillating waveform, a multistage variable gain amplifier for amplifying the oscillating waveform to generate an output signal for selecting a mode of operation for the surgical instrument, and an electrosurgical connector for transmitting the output signal to one or more electrodes on the surgical instrument, wherein the controller modulates the frequency to reduce the at least one of the phase angle and the reactance of the tissue to maximize power output to the tissue.

In accordance with yet another embodiment, an electrosurgical generator for controlling a surgical instrument is provided. The electrosurgical generator includes a controller programmed to generate a first carrier wave signal and a second carrier wave signal based on an algorithm executed on a processor employing real-time current values of tissue at a tissue site to determine tissue impedance at a distal end of the surgical instrument, the first carrier wave signal having a first oscillating waveform and the second carrier wave signal having a second oscillating waveform, a multistage variable gain amplifier for amplifying the first and second oscillating waveforms to generate a first output signal for a first mode of operation and a second output signal for a second mode of operation, and an electrosurgical connector for transmitting the first and second output signals to one or more electrodes on the surgical instrument, wherein the controller runs the first and second oscillating waveforms in an alternating manner while switching between the first mode of operation and the second mode of operation.

It should be noted that the exemplary embodiments are described with reference to different subject-matters. In particular, some embodiments are described with reference to method type claims whereas other embodiments have been described with reference to apparatus type claims. However, a person skilled in the art will gather from the above and the following description that, unless otherwise notified, in addition to any combination of features belonging to one type of subject-matter, also any combination between features relating to different subject-matters, in particular, between features of the method type claims, and features of the apparatus type claims, is considered as to be described within this document.

These and other features and advantages will become apparent from the following detailed description of illustrative embodiments thereof, which is to be read in connection with the accompanying drawings.

### BRIEF DESCRIPTION OF THE DRAWINGS

The invention will provide details in the following description of preferred embodiments with reference to the following figures wherein:

FIG. 1 is an exemplary block/flow diagram of a bipolar electrosurgical generator architecture, in accordance with an embodiment of the present invention;

FIG. 2 is an exemplary block/flow diagram of a monopolar electrosurgical generator architecture, in accordance with an embodiment of the present invention;

FIG. 3A is an exemplary graph of a power output signal having a rectangular waveform envelope, in accordance with an embodiment of the present invention;

FIG. 3B is an exemplary graph of a power output signal having a dual frequency rectangular waveform envelope, in accordance with an embodiment of the present invention;

FIG. 4A is an exemplary graph of a power output signal having a rectangular waveform envelope with a rectangular pulse wave, in accordance with an embodiment of the present invention;

FIG. 4B is an exemplary graph of a power output signal having a dual frequency rectangular waveform envelope with a rectangular pulse wave, in accordance with an embodiment of the present invention;

FIG. 5A is an exemplary graph of a power output signal having a rectangular waveform envelope with a rectangular high and low pulse wave, in accordance with an embodiment of the present invention;

FIG. 5B is an exemplary graph of a power output signal having a rectangular waveform envelope with a diamond pulse wave, in accordance with an embodiment of the present invention;

FIG. 6A is an exemplary graph of a power output signal having a rectangular waveform envelope with an elliptical pulse wave, in accordance with an embodiment of the present invention;

FIG. 6B is an exemplary graph of a power output signal having a rectangular waveform envelope with a ramp up pulse wave, in accordance with an embodiment of the present invention;

FIG. 7 is an exemplary block/flow diagram of an electrosurgical connector connected to a surgical instrument via a connector system, the surgical instrument configured to communicate with an Internet-of-Things (IoT) network, in accordance with an embodiment of the present invention;

FIG. 8 are exemplary views of a radio frequency identification (RFID) bipolar and monopolar receptacle and plug, in accordance with an embodiment of the present invention;

FIG. 9 is an exemplary block/flow diagram of a smart medical environment employing the electrosurgical generator architectures of FIGS. 1 and 2, in accordance with an embodiment of the present invention; and

FIG. 10 is an exemplary block/flow diagram of a plurality of electrosurgical connectors communicating with each other in a smart medical environment, in accordance with an embodiment of the present invention.

Throughout the drawings, same or similar reference numerals represent the same or similar elements.

### DETAILED DESCRIPTION

Embodiments in accordance with the present invention provide exemplary electrosurgical generators that produce a real-time high-frequency modulation signal to regulate power output for an electrosurgical instrument. The exemplary electrosurgical generators generate a modulating frequency carrier signal used to regulate power output for an electrosurgical instrument including microcontrollers programmed to generate a carrier signal having a modulating frequency and to switch the carrier signal between a number of ON and OFF times to create individual energy pulses of the waveform. The energy pulses can be constant per power output or they can vary in duration depending on impedance, phase angle, and/or reactance. The exemplary electrosurgical generators further have 4-stage amplification in communication with the electrical waveform that amplifies the waveform, a radio frequency (RF) filter module to remove harmonics, impedance matching to create a matched power output signal to each load and a receptacle configured to receive an electrosurgical instrument and to pass the electrical signal to the electrosurgical instrument. Multiple feedback circuits in continuous real-time communication with sensing circuits positioned in the exemplary electrosurgical generators receive, e.g., electrical signals and temperature data from the operative field and the exemplary electrosurgical generators make adjustments to the, e.g., frequency, power supply, amplifier, and capacitors based on power output data.

Embodiments in accordance with the present invention provide exemplary electrosurgical generators that produce a real-time high-frequency modulation signal to regulate power output for an electrosurgical instrument by varying the frequency to match the ideal frequency to each tissue (impedance), thus controlling the power output and keeping the tissue temperature at a low level. For example, during coagulation mode, the unit will modulate in a lower frequency range to control the depth of penetration and improve blood vessel sealing. Additionally, the power output can be regulated based on a number of measured parameters and/or variables.

It is to be understood that the present invention will be described in terms of a given illustrative architecture; however, other architectures, structures, substrate materials and process features and steps/blocks can be varied within the scope of the present invention. It should be noted that certain features cannot be shown in all figures for the sake of clarity. This is not intended to be interpreted as a limitation of any particular embodiment, or illustration, or scope of the claims.

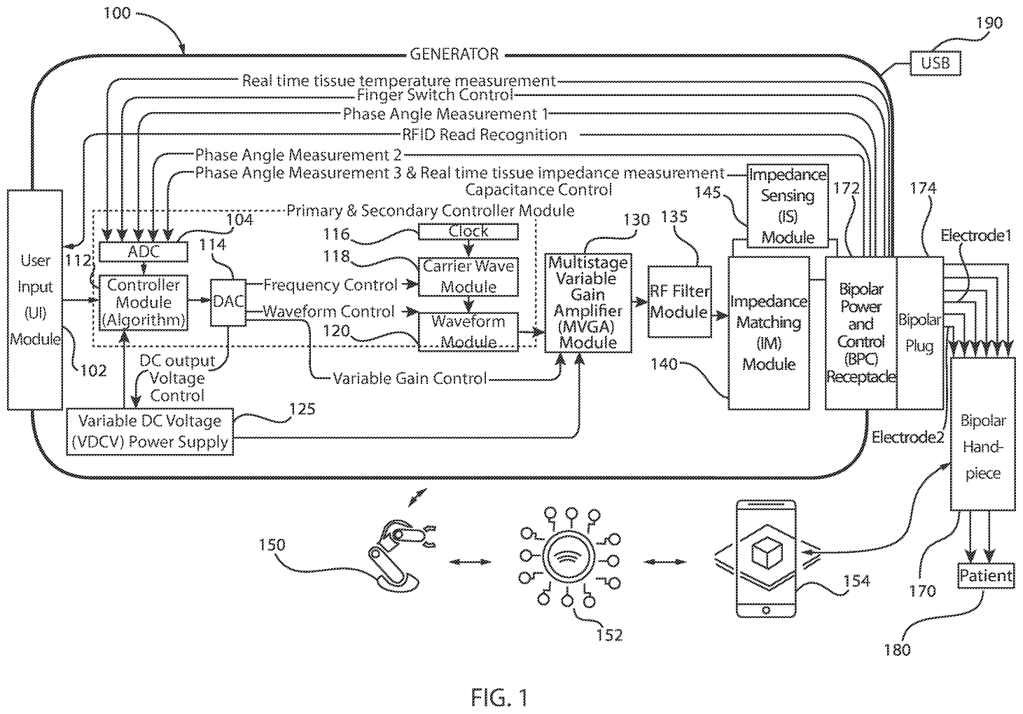

FIG. 1 is an exemplary block/flow diagram of a bipolar electrosurgical generator architecture, in accordance with an embodiment of the present invention.

The electrosurgical generator 100 includes a user input (UI) module 102 that communicates with a primary and secondary controller (PSC) module 110. The PSC module 110 includes an analog-to-digital converter (ADC) 104, a controller module 112, a digital-to-analog converter (DAC) 114, a clock 116, a carrier wave module 118, and a waveform module 120. The inputs received by the UI module 102 are provided to the controller module 112 of the PSC module 110.

The PSC module 110 can be programmed to generate a pure sine carrier wave signal whose frequency is then modulated between about 200 kHz to about 10 MHz based on an algorithm utilizing a real-time tissue impedance reading in communication with the impedance sensing (IS) module 145 and then to switch the carrier wave signal between ON times and OFF times at a frequency between about 1 Hz and about 1000 Hz, and with an ON time duration between about 0.05 milliseconds to about 1000 milliseconds and with OFF time duration between about 0.001 milliseconds to about 10 milliseconds to create an inactive waveform signal to allow communication of digital and analog data between the PSC module 110 and other modules during the OFF time to eliminate interference from the high frequency power output during the ON times. The inactive waveform signal is created when the first carrier wave signal and the second carrier wave signal are in an OFF state to allow communication between at least the controller 112, the multistage variable gain amplifier 130, an impedance matching module 140, and radio frequency identification (RFID) elements described below.

The PSC module 110 switches the carrier wave signal between ON times and OFF times to create repeating energy pulses and a switching frequency of the ON time and OFF times is between about 1 kHz and about 200 kHz with a duty cycle of between about 1% to 95%. The discrete energy pulses of the waveform can be formed within a pulse shape envelope having a shape of one of a group of rectangular, square, triangular, diamond, saw tooth, non-uniform, stair-step, ramp up, rectified ramp up, ramp down, rectified ramp down, sine, circle, elliptical, oval and random or any distorted version of the intended waveforms envelope shapes or an effect caused by an induced transient oscillation.

The output of the PSC module 110 is received by a multi-stage variable gain amplifier (MVGA) module 130, which feeds an impedance matching (IM) module 140 via a radiofrequency (RF) filter 135. The electrosurgical generator 100 further includes an impedance sensing (IS) module 145.

The MVGA module 130 is capable of receiving the waveform signal and amplifying and isolating the waveform signal in successive stages. The MVGA module 130 is in electrical connection with the PSC module 110 for adjusting the amplitude of the waveform signal from a power setting entered into the UI module 102.

The MVGA module 130 is in electrical connection with the IS module 145 and reads the real-time tissue impedance and inputs that data into the algorithm 112 that executes a command that adjusts the variable gain amplifier to control the amplitude of the waveform signal to regulate the output power.

The IM module 140 is in electrical communication with the MVGA module 130 to receive a waveform signal and maximize the output power per input tissue impedance.

The IM module 140 is in electrical communication with the PSC module 110 and reads the real-time tissue impedance and inputs that data into the algorithm 112 that executes a command that adjusts a capacitance of the IM module 140 to optimize the impedance matching per load, to regulate the output power, and to adjust the inductance by measuring the current with the IM module.

The IM module 140 can perform real-time analysis of tissue impedance from a return line in monopolar just inside the monopolar power and control receptacle and either of the two bipolar lines just inside the bipolar power and control receptacle executing the algorithm 112 to estimate real-time tissue impedance at hand piece tip. The unit will measure and record data points of impedance with different loads from 0 to about 2000 ohms by using the algorithm 112 to calculate the impedance correction factor to equal the actual impedance/measured impedance. The impedance correction factor can be determined by measuring and recording impedance data points at the load with the neutral electrode, monopolar electrode, and bipolar electrodes from 0 to about 2000 ohms to obtain the actual impedance. The actual impedance is divided that with the same measurements taken inside the receptacles to obtain the measured impedance. The impedance correction factor is then programmed during the calibration phase. During real-time tissue impedance measurements, the unit multiplies the measured impedance*impedance correction factor to get the actual impedance.

The IS module 145 is in electrical communication with either line of the BPC receptacle 172 and is located in very close proximity to the inside of the receptacle. The IS module 145 measures the real-time current values at the receptacle and employs the algorithm 112 that calculates the impedance at the tip of the monopolar and bipolar electrosurgical instruments 210, 172.

The electrosurgical generator 100 is connected to, e.g., a bipolar hand piece 170 that is employed to treat a patient 180. The electrosurgical generator 100 communicates with the bipolar hand piece 170 via a bipolar power and control (BPC) receptacle 172 and a bipolar plug 174. The BPC receptacle 172 is configured to receive a bipolar electrosurgical instrument and to pass the output waveform signal to the bipolar electrosurgical instrument.

The ADC 104 is configured to receive signals from the bipolar hand piece 170 via the BPC receptacle 172. The signals received by the ADC 104 can include, but are not limited to, e.g., real-time tissue temperature measurements, finger switch control data, phase angle measurements, radio frequency identification (RFID) read recognition data, and real-time tissue impedance measurements. The output of the electrosurgical generator 100 can be regulated based on any of these factors or a combination of these factors.

The ADC 104 converts these analog signals into digital data to be provided to the controller module 112, which includes an algorithm. The controller module 112 processes the data and transmits frequency control data to a carrier wave module 118 and waveform control data to a waveform module 120 via the DAC 114. The controller module 112 also transmits variable gain control data to the MVGA module 130. Additionally, the controller module 112 further transmits DC output voltage control data to a variable DC voltage (VDCV) power supply 125.

The VDCV power supply 125 is in electrical communication with the PSC module 110 and reads the real-time tissue impedance and inputs that data into the algorithm 112 that executes a command that adjusts the output DC voltage of the VDCV power supply 125 that feeds a DC voltage to a metal oxide semiconductor field effect transistor (MOSFET) to optimize the power output constant.

It is noted that, in one exemplary embodiment, the electrosurgical generator 100 can be controlled by robotics or one or more robots 150. The one or more robots 150 can operate in an Internet-of-Things (IoT) environment 152. Moreover, the bipolar hand piece 170 can include one or more augmented reality (AR) sensors for measuring a plurality of variables. Data related to the plurality of variables can be displayed on AR-enhanced displays 154, such as tablets, smart phones, computer screens, etc. Thus, the electrosurgical generator 100 of the present invention can be controlled by robotics 150, can operate in an IoT environment 152, and the data generated can be augmented by AR applications 154.

In summary, the electrosurgical generator 100 generates a modulating frequency carrier signal used to regulate power output for an electrosurgical instrument including microcontrollers programmed to generate a carrier signal having a modulating frequency and to switch the carrier signal between a number of ON and OFF times to create individual energy pulses of the waveform. The energy pulses can be constant per power output or they can vary in duration depending on impedance. The electrosurgical generator 100 further has 4-stage amplification in communication with the electrical waveform that amplifies the waveform, an RF filter module to remove harmonics, impedance matching to create a matched power output signal to each load and a receptacle configured to receive an electrosurgical instrument and to pass the electrical signal to the electrosurgical instrument. Multiple feedback circuits in continuous real-time communication with sensing circuits positioned in the electrosurgical generator 100 receive electrical power and temperature data from the operative field and the electrosurgical generator 100 determines adjustments to the, e.g., frequency, power supply, amplifier, and capacitors based on power output data.

In another exemplary embodiment, the PSC module 110 is programmed to generate two different pure sine carrier wave frequencies between about 200 kHz to about 10 MHz. The two carrier wave signals can be generated simultaneously or alternating at a frequency between about 0.1 Hz and about 200 kHz, and a duty cycle between about 1% to 99%. The exemplary electrosurgical generator 100 can thus generate two frequencies to deliver energy either at the same time or alternating (parallel or series). For example, in one instance the wave signals are generated simultaneously, 800 kHz (25 watts) and 3 MHz (75 watts)=100 watts to control the amplitude and the duty cycle.

In another exemplary embodiment, depth of energy penetration in tissue control can be achieved by a hybrid frequency or by high frequency division multiplexing (HFDM). In such case, an oscillating waveform can be controlled that alternates frequency between about 200 kHz-10 MHz at about 1-99% and about 1-5 MHz at 1-99% to control depth of energy penetration. For example, in one instance, 200 kHz at 25% and 4 MHz at 75%, which provides an advantage for cut, ablation, blend, coagulation, hemostasis, and denervation. This is achieved by generating two carrier frequencies (e.g., 4 MHz and 400 kHz) simultaneously modulating the power output of each. The total bandwidth is divided into non-overlapping frequency bands to deliver a hybrid power signal to the electrosurgical instrument.

In another exemplary embodiment, the MVGA module 130 executes a command that adjusts the variable gain amplifier to control the amplitude of the each of the two different waveform signals to regulate the output power and control the depth of penetration.

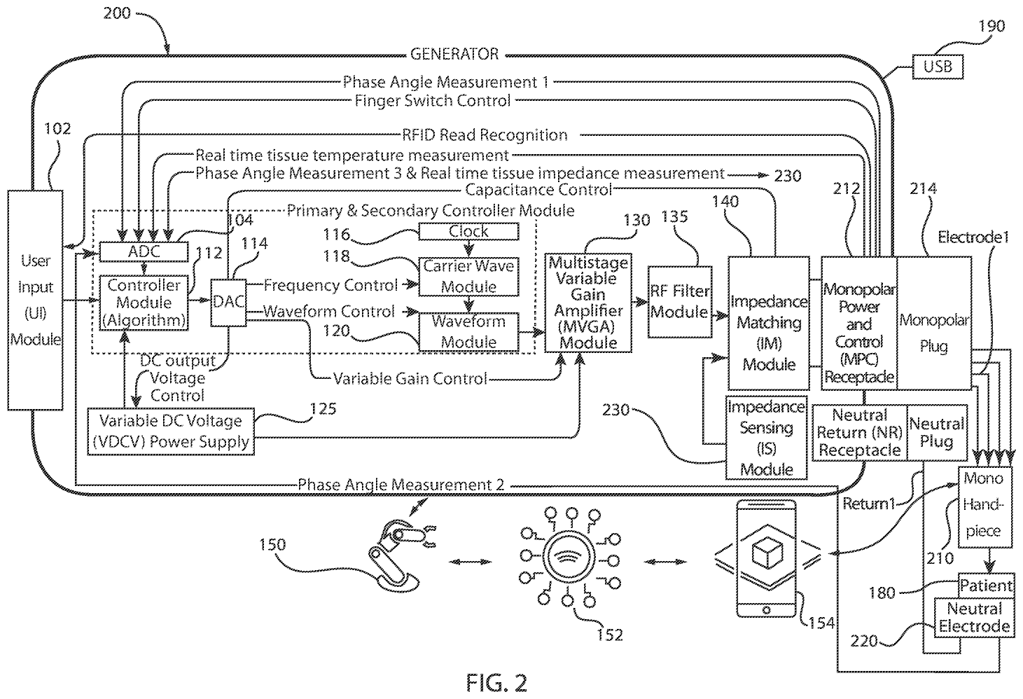

In another exemplary embodiment, the IM module 140 includes a switching mechanism that alternates the output waveform signal between e.g., 4 output pins in the MPC receptacle 212 (FIG. 2) at a frequency of about 1 to about 10,000 switches per second. In another exemplary embodiment, the output pins can be more than 4 pins. One skilled in the art can contemplate a higher or lower number of output pins. This allows for alternating delivery of the energy to different electrode heads in an electrosurgical instrument. This provides an advantage in distributing the energy to different areas of the tissue and keeping the tissue temperatures cooler.

In another exemplary embodiment, the PSC module 110 is programmed to have an OFF time duration between about 0.001 milliseconds to about 10 milliseconds to create an inactive waveform signal to allow the switching mechanism to make contact without sparking and causing wear and tear to the contacts.

FIG. 2 is an exemplary block/flow diagram of a monopolar electrosurgical generator architecture, in accordance with an embodiment of the present invention.

Similarly to FIG. 1, the electrosurgical generator 200 includes a user input (UI) module 102 that communicates with a primary and secondary controller (PSC) module 110. The PSC module 110 includes an analog-to-digital converter (ADC) 104, a controller module 112, a digital-to-analog converter (DAC) 114, a clock 116, a carrier wave module 118, and a waveform module 120. The inputs received by the UI module 102 are provided to the controller module 112 of the PSC module 110.

The output of the PSC module 110 is received by a multi-stage variable gain amplifier (MVGA) module 130, which feeds an impedance matching (IM) module 140 via a radiofrequency (RF) filter 135. The electrosurgical generator 200 further includes an impedance sensing (IS) module 230.

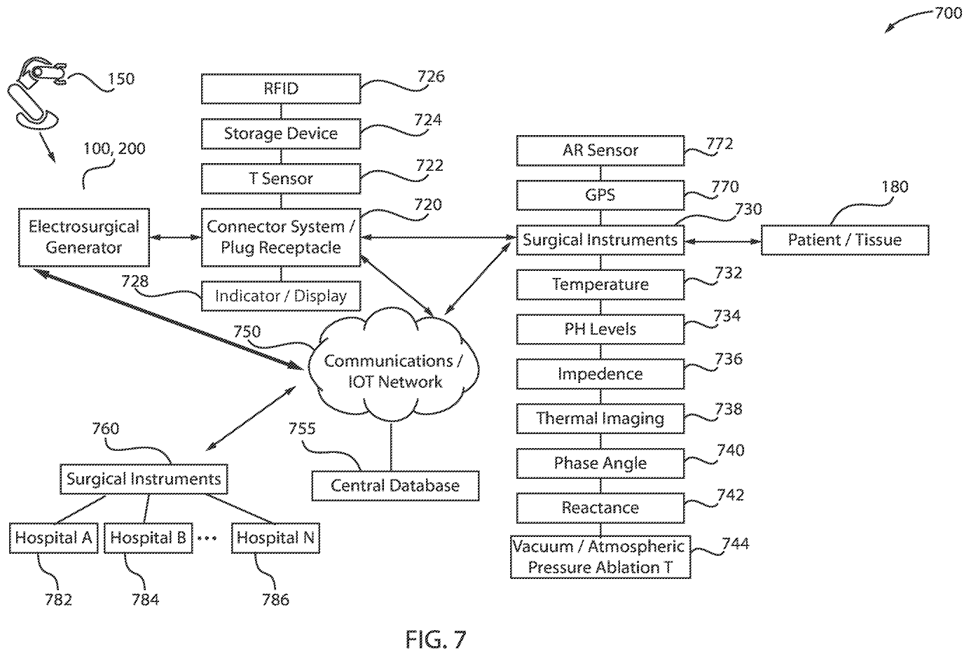

The electrosurgical generator 200 is connected to, e.g., a monopolar hand piece 210 that is employed to treat a patient 180. The patient 180 can have a neutral electrode 220 attached thereto. The electrosurgical generator 200 communicates with the monopolar hand piece 210 via a monopolar power and control (MPC) receptacle 212 and a monopolar plug 214. The MPC receptacle 212 is configured to receive a monopolar electrosurgical instrument and to pass the output waveform signal to the monopolar electrosurgical instrument. The MPC receptacle 212 communicates with a neural return (NR) receptacle 218 and the monopolar plug 214 communicates with a neutral plug 216. An electrosurgical connector 720 (FIG. 7) can transmit first and second output signals to one or more electrodes on the surgical instrument 170, 210 (FIGS. 1, 2). In one instance, one electrode can be on the surgical instrument 170, 210 with a 4 MHz output signal and another electrode can be on the surgical instrument 170, 210 with a 1 MHz output signal. Of course, one skilled in the art can contemplate different frequencies for the output signals.

As noted above, in one exemplary embodiment, the electrosurgical generator 200 can be controlled by robotics or one or more robots 150. The one or more robots 150 can operate in an Internet-of-Things (IoT) environment 152. Moreover, the monopolar hand piece 210 can include one or more augmented reality (AR) sensors for measuring a plurality of variables. Data related to the plurality of variables can be displayed on AR-enhanced displays 154, such as tablets, smart phones, computer screens, etc. Thus, the electrosurgical generator 200 of the present invention can be controlled by robotics 150, can operate in an IoT environment 152, and the data generated can be augmented by AR applications 154.

The exemplary electrosurgical generators 100, 200 of FIGS. 1 and 2 read temperature from an electrosurgical instrument plugged thereto and vary the modules to regulate power output and also regulate the tissue temperature by varying the variable gain amplifier, by varying the Variable DC voltage (VDCV) power supply 125, by varying capacitance, and/or by varying frequency.

In particular, the electrosurgical instruments 170, 210 can include at least one temperature sensing device, the temperature sensing device configured to collect temperature signals from the electrosurgical instrument that represents tissue temperature from an operative field.

The electrosurgical instruments 170, 210 can further include a feedback circuit to the PSC module 110 in electrical connection and communication with the temperature sensing device that reads the real-time tissue temperatures and inputs that data into the algorithm 112 that executes a command that adjusts the MVGA module 130 to control the amplitude of the waveform signal to regulate the output power and to regulate the tissue temperature.

The electrosurgical instruments 170, 210 can further include a feedback circuit to the PSC module 110 in electrical connection and communication with the temperature sensing device that reads the real-time tissue temperatures and inputs that data into the algorithm 112 that executes a command that adjusts the output DC voltage of the VDCV power supply 125 that feeds a DC voltage to the MOSFET to optimize the power output and to regulate the tissue temperature.

The electrosurgical instruments 170, 210 can further include a feedback circuit to the PSC module 110 in electrical connection and communication with the temperature sensing device that reads the real-time tissue temperatures and inputs that data into the algorithm 112 that executes a command that adjusts the capacitance of the IM module 140 to optimize the impedance matching per load, to regulate the output power, and to regulate the tissue temperature.

The electrosurgical instruments 170, 210 can further include a feedback circuit to the PSC module 110 in electrical connection and communication with the temperature sensing device that reads the real-time tissue temperatures and inputs that data into the algorithm 112 that executes a command whose carrier wave frequency is then modulated between about 200 kHz to about 10 MHz to regulate the output power and to regulate the tissue temperature.

The exemplary electrosurgical generators 100, 200 of FIGS. 1 and 2 regulate the power output during, e.g., ablation, cutting, coagulation, and denervation by monitoring and analyzing real-time tissue impedance and executing an algorithm to:

By varying frequency: to control the carrier wave frequency between about 200 kHz and about 10 MHz to optimize the power output. By varying the frequency, the exemplary embodiments can optimize the impedance matching circuit and generate a higher output per tissue impedance.

By varying capacitance: to control the capacitance of an LC circuit in the impedance matching circuit to optimize the power output. This increases the efficiency of the impedance matching circuit and maximizes the power output vs the tissue impedance.

By varying the variable gain amplifier: to control the variable gain amplifier to optimize the power output.

By varying the power supply: to control the output DC voltage of the power supply that feeds a DC voltage to the MOSFET to optimize the power output.

The exemplary electrosurgical generators 100, 200 of FIGS. 1 and 2 regulate the tissue temperature during, e.g., ablation, cutting, coagulation, and denervation by monitoring and analyzing real-time tissue temperature and executing an algorithm to:

By varying frequency: to control the carrier wave frequency between about 200 kHz and about 10 MHz to optimize the power output.

By varying capacitance: to control the capacitance of an LC circuit in the impedance matching circuit to optimize the power output. This increases the efficiency of the impedance matching circuit and maximizes the power output vs the tissue impedance.

By varying the variable gain amplifier: to control the variable gain amplifier to optimize the power output.

By varying the power supply: to control the output DC voltage of the power supply that feeds a DC voltage to the MOSFET to optimize the power output.

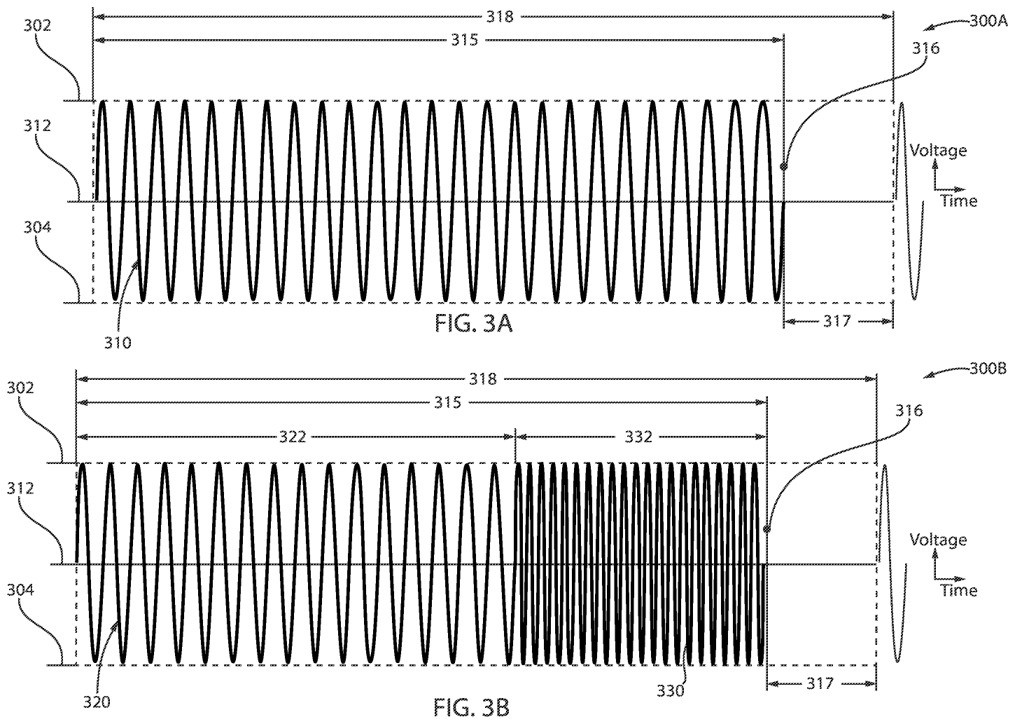

FIG. 3A is an exemplary graph 300A of a power output signal having a rectangular waveform envelope, in accordance with an embodiment of the present invention.

The frequency generation module generates a pure sine wave. The frequency of the oscillating wave is then modulated between about 200 kHz to about 10 MHz based on real-time tissue impedance readings to form the carrier wave 310. The carrier wave 310 is then switched OFF with a waveform OFF time 317 to create an inactive waveform signal to allow communication of digital and analog data between the modules.

Moreover, the monopolar electrosurgical instrument 210 can include up to three finger switch buttons each connected to the device to select and activate one of the monopolar modes and deliver a unique waveform signal. Similarly, the bipolar electrosurgical instrument can include up to three finger switch buttons each connected to the device to select and activate one of the monopolar modes and deliver a unique waveform signal.

Further, a monopolar footswitch can be provided with first, second, and third switches each connected to the device to select and activate one of the monopolar and bipolar modes and deliver a unique waveform signal.

Similarly, a bipolar footswitch can be provided with first, second, and third switches each connected to the device to select and activate one of the bipolar modes and deliver a unique waveform signal.

The carrier wave 310 is a pure sine wave whose frequency is modulated from about 200 kHz to about 10 MHz dependent on tissue impedance readings. The upper envelope signal 302 is a positive peak voltage, Vₚ. The lower envelope signal 304 is a negative peak voltage, Vₚ. The baseline signal 312 is approximately zero voltage and is considered the crossover point. The waveform on time 315 is the number of pulse wave cycles generated in one waveform cycle. Regarding the waveform envelope shape 316, this waveform envelope can be any shape or any distorted version of the intended waveforms envelope shapes. 317 designates the waveform OFF time. The advantage of the OFF time is communication of digital and analog signals between modules without interferences from the high-frequency power output signal during the ON time. The waveform cycle 318 is one cycle or period (t) of the waveform, which is about 1 Hz to about 200 Hz. The period of a wave is the amount of time it takes to complete one cycle.

FIG. 3B is an exemplary graph 300B of a power output signal having a dual frequency rectangular waveform envelope, in accordance with an embodiment of the present invention.

The frequency generation module generates a pure sine wave. The frequency of the oscillating wave is then modulated between about 200 kHz to about 10 MHz based on real-time tissue impedance readings to form the carrier wave 320. The carrier wave 320 is then switched OFF and the frequency generation module generates a pure sine wave. The frequency of the oscillating wave is then modulated between about 200 kHz to about 10 MHz based on real-time tissue impedance readings to form the carrier wave 330 with a waveform OFF time 317 to create an inactive waveform signal to allow communication of digital and analog data between the modules.

The first carrier wave signal 320 is a pure sine wave whose frequency is modulated from about 200 kHz to about 10 MHz dependent on tissue impedance readings. The second carrier wave signal 330 is pure sine wave whose frequency is modulated from about 200 kHz to about 10 MHz dependent on tissue impedance readings. The first carrier wave signal 320 has an ON time 322 of the carrier wave with a first frequency. The second carrier wave signal 330 has an ON time 332 of the carrier wave signal with a second frequency.

The controller 112 of FIGS. 1 and 2 can concurrently run the first and second oscillating waveforms while switching between a first mode of operation and a second mode of operation. A frequency of the first carrier wave signal and a frequency of the second carrier wave signal are modulated by employing the algorithm to optimize the frequencies of the first and second carrier wave signals based on the determined tissue impedance. The first oscillating waveform and the second oscillating waveform are formed within a rectangular modulation envelope, the first oscillating waveform forming first energy pulses of a first shape and the second oscillating waveform forming second energy pulses of a second shape. Moreover, the rectangular modulation envelope remains constant regardless of the first shape of the first energy pulse and the second shape of the second energy pulses formed therein. In other words, the rectangular modulation envelope is not defined by the first shape of the first energy pulses and the second shape of the second energy pulses formed therein.

Regarding FIGS. 3A-3B, the purpose of the OFF time of the waveform is to allow communication of digital and analog data between the primary and secondary microcontroller module (PSMM) and other modules. During the OFF time, it eliminates interference from the high frequency power output during the ON state.

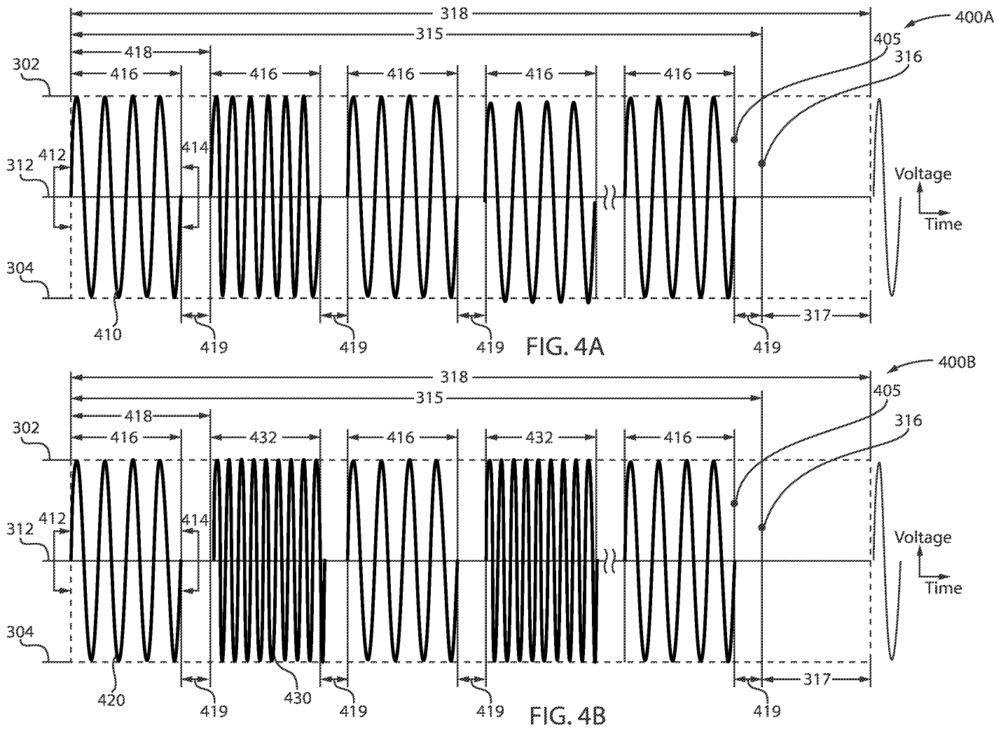

FIG. 4A is an exemplary graph 400A of a power output signal having a rectangular waveform envelope with a rectangular pulse wave, in accordance with an embodiment of the present invention.

The frequency generation module generates a pure sine wave. The frequency of the oscillating wave is then modulated between about 200 kHz to about 10 MHz based on real-time tissue impedance readings to form the carrier wave 410. The carrier wave 410 is then modulated with a digital pulse wave input signal to create a repeating pulse wave cycle 418. The carrier wave 410 is modulated to match the rising edge 412 and falling edge 414 to form the pulse wave rectangular shape 405. The carrier wave 410 is then switched OFF with a waveform OFF time 317 to create an inactive waveform signal to allow communication of digital and analog data between the modules. Squiggly line shows that number of pulses per waveform in one frame is not accurate representation.

The rising edge 412 is a transition from low to high, whereas the falling edge 414 is a transition from high to low. The pulse ON time 416 defines the pulse width and the pulse OFF time 419 may be constant from OFF time to OFF time or it may randomly vary from OFF time to OFF time. The pulse wave shape 405 is a diamond or any distorted version of the intended pulse wave shape. The pulse wave cycle 418 is one cycle or period of a pulse, which is equal to about 20 kHz to about 200 kHz.

FIG. 4B is an exemplary graph 400B of a power output signal having a dual frequency rectangular waveform envelope with a rectangular pulse wave, in accordance with an embodiment of the present invention.

The frequency generation module generates a pure sine wave. The frequency of the oscillating wave is then modulated between about 200 kHz to about 10 MHz based on real-time tissue impedance readings to form the carrier wave 420. The carrier wave 420 is then modulated with a digital pulse wave input signal to create a repeating pulse wave cycle 418. The carrier wave 420 is modulated to match the rising edge 412 and falling edge 414 to form the pulse wave rectangular shape 405. The carrier wave 420 is then switched OFF with a waveform OFF time 317 to form carrier waveform 430 to create an inactive waveform signal to allow communication of digital and analog data between the modules. The first carrier wave signal 420 has an ON time 416 of the carrier wave with a first frequency. The second carrier wave signal 430 has an ON time 432 of the carrier wave signal with a second frequency. Squiggly line shows that number of pulses per waveform in one frame is not accurate representation.

The pulse ON times 416, 432 define the pulse widths of signals 420, 430, and the pulse OFF time 419 may be constant from OFF time to OFF time or it may randomly vary from OFF time to OFF time. The pulse wave shape 405 is a diamond or any distorted version of the intended pulse wave shape. The pulse wave cycle 418 is one cycle or period of a pulse, which is equal to about 20 kHz to about 200 kHz.

Regarding FIGS. 4A-4B, for the pulse wave modulation, the purpose is to control OFF time vs ON time of the power signal that regulates depth of energy penetration in tissue to optimize shrinking of blood vessels and nerve fibers. This creates individual energy pulses of the waveform. The PWM frequency can be constant or modulate.

Regarding FIGS. 4A-4B, for the waveform modulation, the purpose of the OFF time of the waveform is to allow communication of digital and analog data between the primary and secondary microcontroller module (PSMM) and other modules. During the OFF time, it eliminates interference from the high frequency power output during the ON state.

Regarding the carrier wave generation, this can be referred to as High Radio Frequency Modulation (HRFM) or Variable High Radio Frequency (VHRF) or Variable High Radio Regulation (VHRR) or Variable Frequency Power Regulation (VFPR) or Frequency Modulation Power Regulation (FMPR) or Carrier Wave frequency modulation (CWFM).

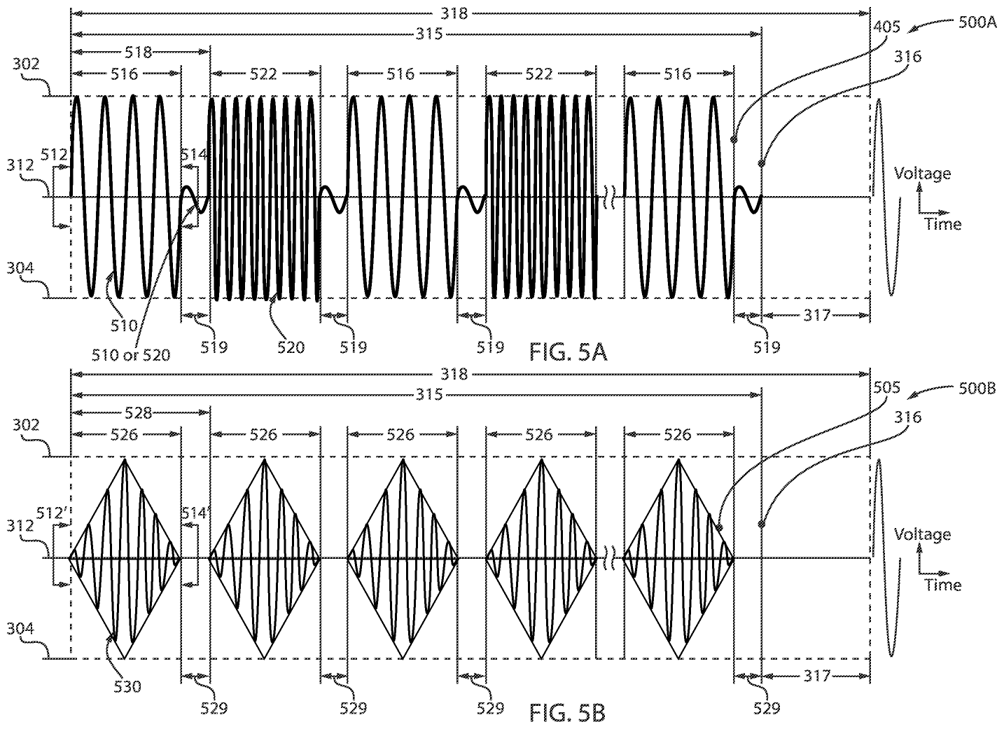

FIG. 5A is an exemplary graph 500A of a power output signal having a rectangular waveform envelope with a rectangular high and low pulse wave, in accordance with an embodiment of the present invention.

The frequency generation module generates a pure sine wave. The frequency of the oscillating wave is then modulated between about 200 kHz to about 10 MHz based on real-time tissue impedance readings to form the carrier wave 510. The carrier wave 510 is then modulated with a digital pulse wave input signal to create a repeating pulse wave cycle 518. The carrier wave 510 is modulated to match the rising edge 512 and falling edge 514 to form the pulse wave rectangular shape 405. The carrier wave 510 is then switched OFF with a waveform OFF time 317 to create an inactive waveform signal to allow communication of digital and analog data between the modules. The first carrier wave signal 510 has an ON time 516 of the carrier wave with a first frequency. The second carrier wave signal 520 has an ON time 522 of the carrier wave signal with a second frequency. Squiggly line shows that number of pulses per waveform in one frame is not accurate representation.

The carrier wave signal 510 or 520 is a pure sine wave whose frequency is between about 200 kHz to about 10 MHz dependent on tissue impedance readings. The pulse LOW time 519 may be constant from LOW time to LOW time or it may randomly vary from LOW time to LOW time. There is a benefit of going from an ON state to a LOW state in that the exemplary embodiments of the present invention can have a longer LOW state duration (lower duty cycle) and this has an improved coagulation effect and shrinking of the intervertebral disc tissue. The variables that can be controlled are LOW state power output and duty cycle to optimize the coagulation and shrinking of intervertebral disc tissue. This is opposed to a shorter OFF state with a higher duty cycle. Therefore, the first carrier wave signal and the second carrier wave signal are switched between an ON state and a LOW state to create high energy pulse waves and low energy pulse waves.

FIG. 5B is an exemplary graph 500B of a power output signal having a rectangular waveform envelope with a diamond pulse wave, in accordance with an embodiment of the present invention.

The frequency generation module generates a pure sine wave. The frequency of the oscillating wave is then modulated between about 200 kHz to about 10 MHz based on real-time tissue impedance readings to form the carrier wave 530. The carrier wave 530 is then modulated with a digital pulse wave input signal to create a repeating pulse wave cycle 528. The amplitude of the carrier wave 530 is modulated to match the rising edge 512′ and falling edge 514′ to form the pulse wave diamond shape 505. The carrier wave 530 is then switched OFF with a waveform OFF time 317 to create an inactive waveform signal to allow communication of digital and analog data between the modules. Therefore, the first carrier wave signal is modulated with a digital pulse wave to create a repeating pulse wave cycle. The pulse ON time 526 defines the pulse width and the pulse OFF time 529 may be constant from OFF time to OFF time or it may randomly vary from OFF time to OFF time. Squiggly line shows that number of pulses per waveform in one frame is not accurate representation.

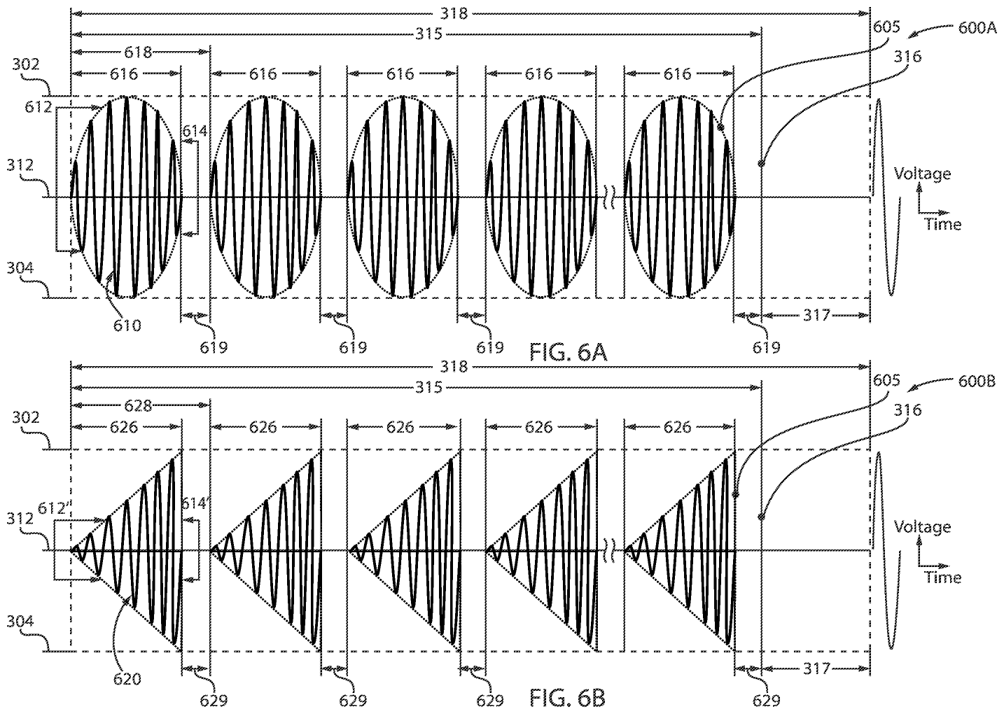

FIG. 6A is an exemplary graph 600A of a power output signal having a rectangular waveform envelope with an elliptical pulse wave, in accordance with an embodiment of the present invention.

The frequency generation module generates a pure sine wave. The frequency of the oscillating wave is then modulated between about 200 kHz to about 10 MHz based on real-time tissue impedance readings to form the carrier wave 610. The carrier wave 610 is then modulated with a digital pulse wave input signal to create a repeating pulse wave cycle 618. The amplitude of the carrier wave 610 is modulated to match the rising edge 612 and falling edge 614 to form the pulse wave elliptical shape 605. The carrier wave 610 is then switched OFF with a waveform OFF time 317 to create an inactive waveform signal to allow communication of digital and analog data between the modules. The pulse ON time 616 defines the pulse width and the pulse OFF time 619 may be constant from OFF time to OFF time or it may randomly vary from OFF time to OFF time. Squiggly line shows that number of pulses per waveform in one frame is not accurate representation.

FIG. 6B is an exemplary graph 600B of a power output signal having a rectangular waveform envelope with a ramp up pulse wave, in accordance with an embodiment of the present invention.

The frequency generation module generates a pure sine wave. The frequency of the oscillating wave is then modulated between about 200 kHz to about 10 MHz based on real-time tissue impedance readings to form the carrier wave 620. The carrier wave 620 is then modulated with a digital pulse wave input signal to create a repeating pulse wave cycle 628. The amplitude of the carrier wave 620 is modulated to match the rising edge 612′ and falling edge 614′ to form the pulse wave ramp up shape 625. The carrier wave 620 is then switched OFF with a waveform OFF time 317 to create an inactive waveform signal to allow communication of digital and analog data between the modules. The pulse ON time 626 defines the pulse width and the pulse OFF time 629 may be constant from OFF time to OFF time or it may randomly vary from OFF time to OFF time. Squiggly line shows that number of pulses per waveform in one frame is not accurate representation.

Regarding FIGS. 3A-6B, the modulation envelope can have a shape of one of a group of rectangular, square, triangular, diamond, saw tooth, non-uniform, stair-step, ramp up, rectified ramp up, ramp down, rectified ramp down, sine, circle, elliptical, oval and random or any distorted version of the intended waveforms envelope shapes or an effect caused by an induced transient oscillation.

FIG. 7 is an exemplary block/flow diagram of an electrosurgical connector connected to a surgical instrument via a connector system, the surgical instrument configured to communicate with an Internet-of-Things (IoT) network, in accordance with an embodiment of the present invention.

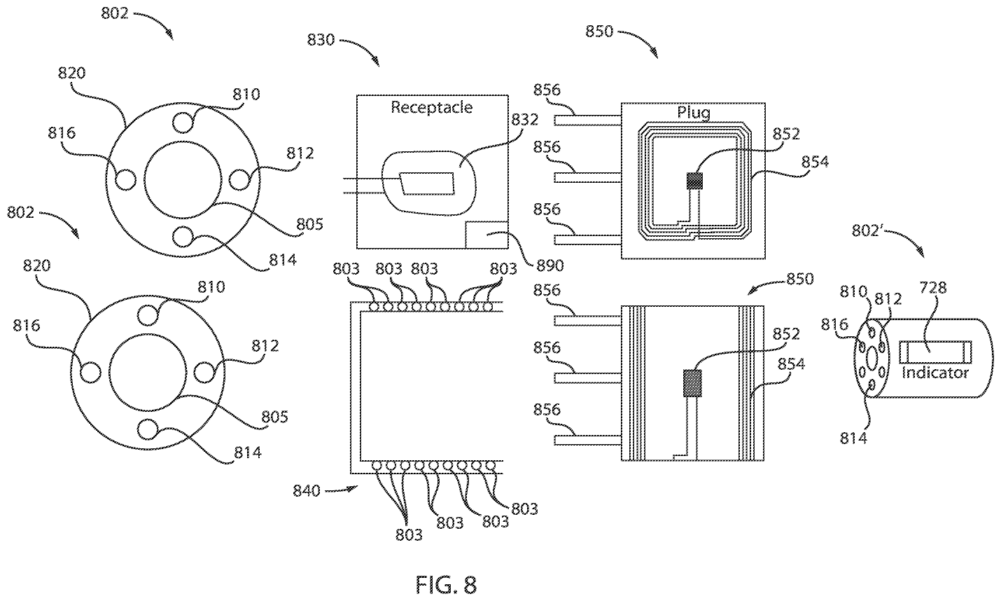

Electrosurgical generators 100, 200 can be connected to a plurality of surgical instruments 730 via electrosurgical connector systems 720 in system 700. The plurality of electrosurgical instruments 730 are used to treat tissue of a patient 180. The connector 720 can be a smart connector that includes a temperature sensor 722, a storage device 724, and an RFID chip 726, as well as an indicator display 728. The temperature sensor 722 can be incorporated in the RFID chip 726 in the plug 850 (FIG. 8). The temperature can be measured at the distal end of the surgical instrument 730. The temperature can be recorded and stored on the storage device 724. A transceiver antenna 832 in the receptacle 830 (FIG. 8) can read the RFID chip 726 in the plug 850 and use such data to control or regulate the power output. Thus, no extra pin are required in the receptacle 830 and the plug 850 for reading the temperature.

In one example embodiment, the electrosurgical generators 100, 200 can be controlled by robotics 150.

Robotics 150 can include, e.g., robotic surgical systems including multiple robotic arms to which a plurality of robotic surgical tools (also referred to as robotic surgical instruments) can be coupled. One such category of robotic surgical tools is electrosurgical tools which includes a monopolar electrosurgical tool or a bipolar electrosurgical tool as well as harmonic, laser, ultrasound tools. Another category of robotic surgical tools is tissue manipulation tools which may have articulated end effectors (such as jaws, scissors, graspers, needle holders, micro dissectors, staple appliers, tackers, suction/irrigation tools, clip appliers, or the like) or non-articulated end effectors (such as cutting blades, irrigators, catheters, suction orifices, or the like) without electrosurgical elements.

The surgical instruments 730 can communicate over an Internet-of-Things (IoT) communication network 750 with a plurality of other surgical instruments 760. The IoT communication network 750 can communicate with a central database 755 for storing generated information. The plurality of other surgical devices 760 can be located, e.g., in a plurality of different hospitals 782, 784, 786. Therefore, information can be received from a plurality of different surgical devices in different rooms of a same hospital and from a plurality of surgical devices from a plurality of different hospitals.

The plurality of surgical instruments 730 can measure or detect various parameters related to tissue, e.g., temperature 732, pH levels 734, impedance 736, thermal imaging 738, phase angle 740, reactance 742, vacuum generation and/or atmospheric pressure 744, etc. The output of the electrosurgical generators 100, 200 can be regulated based on any of these factors or a combination of these factors. The plurality of surgical instruments 730 can also include GPS 770, as well as augmented reality (AR) sensors 772.

pH levels 734 of the tissue at the tissue site can be measured to regulate the output power. When the pH levels 734 of the tissue at the tissue site exceed one or more thresholds, the algorithm 112 (FIG. 1) can terminate one or more power delivery modes. For example, in tissue ablation of cancer cells, there tends to be a lower pH than normal cells. Another example of tissue ablation of the nucleus pulposus in degenerative intervertebral discs lactate is produced and the pH is lower than other surrounding tissues. In one instance, low back pain is known to be related to intervertebral disc degeneration. The algorithm 112 can stop ablation and shrinking of the tissue once it reaches a certain pH level. Thus, precisely ablating and shrinking all the unhealthy tissue with a low pH can be achieved.

Thermal imaging 738 can include a thermal camera attached to an endoscope or other surgical instrument to read temperature gradients. The temperature gradients can be used to control power output of the electrosurgical generators 100, 200 via the algorithm in controller 112. In other words, a thermal camera is in communication with the electro surgical generators 100, 200 to measure temperature gradients at the tissue site to be used to regulate output power.

Phase angle (PA) is the tan value of the ratio of reactance versus electric resistance. PA depends on cell membrane integrity and body cell mass. There exists a correlation between PA values and body cell mass. A high PA shows good health of the tissue, and a low PA shows a worse status of health of the tissue. The phase angle φ is the shift between AC current and voltage. The expression for the phase angle φ is: φ=arctg X/R. Reactance reflects the body cell mass, and the resistance reflects the water or fluid in the body.

The PA or reactance measurement will be taken with the same output frequency of the generator since the reactance is proportional to the frequency. As the output frequency modulates from 200 kHz to 10 MHz, the PA and reactance will change. Thus, it is important that such variables are measured at an accurate frequency.

To determine the phase angle, the electrosurgical generator 100, 200 measures the current and voltage. It then compares a voltage waveform to that of a current waveform to determine the phase angle. In FIGS. 1 and 2, the phase angle measurement 1 and the phase angle measurement 2 determine the voltage at the frequency delivered, and the phase angle measurement 3 determines the current at the frequency delivered. It is noted that different tissues have different reactance and phase angles. The controller 112 reads, in real-time, the reactance and phase angle of the tissue, and then modulates the frequency based on such tissue measurements.

In one or more exemplary embodiments, a vacuum is created at the surgical instrument tip to drop the atmospheric pressure to reduce the boiling point of water, thus lowering the temperature of ablation. Ablation works by heating water molecules to 100 degrees C. where they burst and tissue is ablated. A cannula enters the intervertebral disc, the disc is injected with saline, and the device is inserted and sealed (e.g., special gasket filter so no air gets in). A vacuum draws out air, but not the saline (e.g., special gasket filter) until one reaches a pressure of e.g., 400 mm/hg (e.g., water boils at about 80 C at that pressure), ablation begins. This could occur through, e.g., an endoscope, cannula and/or catheter.

The surgical instruments 730, in their communication, can provide global positioning system (GPS) coordinates of the electrosurgical generator 100, 200 and/or the surgical instruments 730 themselves, as well as a timestamp. The RFID chip 726 and/or electrosurgical generator 100, 200 measure and record impedance, phase angle of tissue, tissue reactance (capacitance and inductance), activation times, serial number, lot number, device manufactured date, expiration date, pressure reading to send for post market clinical follow up data. The electrosurgical generator 100, 200 is able to get calibrated remotely and firmware uploaded through the IOT network 750. For example, a universal serial bus (USB) connection 190 (FIGS. 1 and 2) can be embedded in the rear of electrosurgical generator 100, 200. The electrosurgical generators 100, 200 can be calibrated in a biomed facility or in a remote facility.

The electrosurgical generator 100, 200 can employ Wi-Fi, ZigBee, Bluetooth, Lo-RaWan, LTE-M (or any cellular wireless data format), IEEE 902.11af (White-Fi) and IEE 802.11ah (HaLow) to transmit data.

FIG. 8 are exemplary views of a radio frequency identification (RFID) bipolar and monopolar receptacle and plug, in accordance with an embodiment of the present invention.

The front view 802 illustrates, e.g., 4 pins 810, 812, 814, 816. However, one skilled in the art can contemplate a different number of pins, e.g., up to 10 pins depending on the variables or parameters being measured. Additionally, an RFID antenna 805 is shown wrapped around the cylindrical surface 820.

The side views illustrate a receptacle 830 with an antenna 832 and a plug 850 with a transmitting antenna 854. In one example configuration, the bipolar or monopolar plug has the integrated transmitting antenna 854 on one side of the plug. The receptacle 830 can include a universal serial bus (USB) 890 and read recognition capabilities. Additionally, the cross-sectional view 840 of the bipolar or monopolar receptacle illustrates a cross-section of receiving antennas 803. The plug 850 includes an RFID chip 852 attached to the transmitting antenna 854. The plug 850 includes connecting portions 856. The RFID tag 850 thus has an integrated antenna 854. The antenna 854 can be an RFID reader loop antenna or an antenna coil.

Additionally, a perspective view 802′ of the connector depicts an indicator 728 incorporated on an outer surface of the connector. The indicator 728 can provide continuous real-time information to a user. The indicator 728 can be a display. Thus, the electrosurgical connector 720, 802′ can include temperature sensors, storage devices, USB ports, and RFID chips embedded or incorporated therein, where the RFID chip 852 has an antenna 854 incorporated on an outer surface thereof.

Moreover, the size of the receiving RFID antenna 854 attached to the RFID chip 852 in the hand piece plug 850 is of importance when the RFID chip is being programmed wirelessly. If one wants to batch program ten surgical instruments with an attached RFID chip that are packaged in one box, then the user needs a large transmitting antenna. However, if the receiving RFID antenna 854 is small or on the antenna is on the chip, it will be virtually impossible to batch program because the user needs to be within a few millimeters away. Thus, the receiving RFID antenna 854 size is important. That is why using as much surface area on the hand piece plug 850 is beneficial. The same principals apply for when the transmitting antenna 832 read the receiving RFID antenna 854. An advantage to this antenna configuration is that the connector will have a longer connection with the plug. The antenna being on the outside surface will have less interference than if it is located between output wires. The antenna can be over molded to be concealed.

In another exemplary embodiment, an antenna stacker or antenna expander can be attached to the receptacle 830. A normal length of an antenna can be about 10 feet. To maximize the efficiency of an antenna, its length needs to be the wavelength/2 or the wavelength/4. At a frequency of about 4 MHz, the wavelength would have to be about 74.95 m long, which is much longer than the 10 feet. An antenna expander or antenna stacker could be connected to an output side of the receptacle 830 with longer traces to extend the length of the antenna. The antenna can be, e.g., a helical antenna or a loop antenna to provide for a longer antenna configuration to maximize efficiency. A similar implementation can be employed to the return electrode. Therefore, the antenna stacker or antenna expander can be employed in both the monopolar and bipolar implementations.

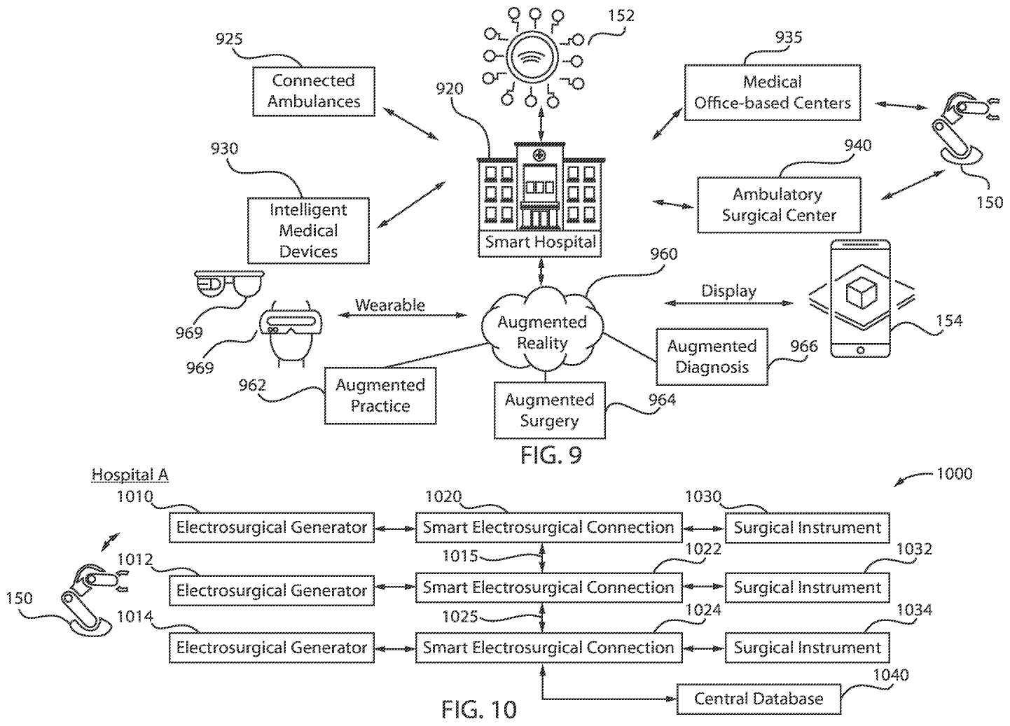

FIG. 9 is an exemplary block/flow diagram of a smart medical environment employing the electrosurgical generator architectures of FIGS. 1 and 2, in accordance with an embodiment of the present invention.

A smart medical environment can include a smart hospital 920 that is connected to an IoT network 152. The smart hospital 920 can communicate with connected ambulances 925 and intelligent medical devices 930, including electrosurgical generators 100, 200. The smart hospital 920 can also communicate with medical office-based centers 935 and ambulatory surgical centers 940 both of which can employ the electrosurgical generators 100, 200. The medical office-based centers 935 and ambulatory surgical centers 940 can both include medical equipment controlled by robotics 150. In other words, robotic surgery can be employed by using the electrosurgical generators 100, 200.

Robotic surgery may be used to perform a wide variety of surgical procedures, including but not limited to open surgery, neurosurgical procedures (such as stereotaxy), endoscopic procedures (such as laparoscopy, arthroscopy, thoracoscopy), and the like. During these robotic surgical procedures, surgeons may use high voltage, low current electrical energy of various wave forms to perform such tasks as cautery, cutting tissue, or sealing a vessel. Electrical energy supply devices (referred to as electrosurgical generating units ESU) are coupled to surgical instruments and typically activated by a foot pedal switch of a foot pedal. One or more foot pedals in a surgeon's console and their corresponding switches may be used to activate these electrical energy supply devices. The foot pedal switches in the surgeon's console replace the original equipment manufacturers (OEM) foot pedal switches that are packaged with the ESUs as standard equipment.

The smart hospital 920 can also be equipped to handle augmented reality applications 960. Augmented reality applications 960 can include augmented practice 962, augmented surgery 964, and augmented diagnosis 966. The augmented reality applications 960 can be implemented by wearable devices 969 or by tablets, smart phones 154, etc.

Regarding augmented surgery 964, as data access technologies are already very advanced, the next step is to provide real-time, life-saving patient information to surgeons which they can use during simple or complex procedures. Augmented reality will allow surgeons to precisely study their patients' anatomy by entering their MRI data and CT scans into an AR headset and overlay specific patient anatomy on top of their body before actually going into surgery. Surgeons will be able to visualize bones, muscles, and internal organs without even having to cut open a body. This could also help them determine exactly where to make injections and incisions and it could be used to display life-saving information for paramedics and first responders during a medical emergency. AR can not only be used to perform accurate and low-risk surgeries, but it can also help surgeons save time in the case of an emergency surgery. Instead of searching among papers or through electronic medical records, surgeons can have access to all of that information on their AR screen within seconds. Additionally, surgeons can regulate the power output of electrosurgical generators 100, 200 based on the AR information.

Regarding augmented diagnosis 966, augmented reality also makes it possible for doctors to better determine their patients' symptoms and accurately diagnose them. Often times patients struggle to accurately describe their symptoms to doctors, but with AR, patients can describe their symptoms better.

Regarding augmented practice 962, the benefits that AR can bring to the field of medicine and education are revolutionary. Medical institutions are beginning to implement AR into their curriculum to provide students with a valuable hands-on learning experiences. Essentially, the idea for using AR in education is to simulate patient and surgical encounters for students to make all of their mistakes on AR rather than in a dissection lab or worse, in a real-life procedure. Students will use AR so they can accurately learn about diagnosing patients with health conditions or take part in an AR surgical procedure. In one example, surgeons and students can regulate the power output of electrosurgical generators 100, 200 based on the AR information, and determine which variables can provide for better power output control.

FIG. 10 is an exemplary block/flow diagram of a plurality of electrosurgical connectors communicating with each other in a smart medical environment, in accordance with an embodiment of the present invention.

In a first hospital 1000, e.g., hospital A, a first electrosurgical generator 1010, a second electrosurgical generator 1012, and a third electrosurgical generator 1014 can be controlled by robotics 150. The first electrosurgical generator 1010 can control a surgical instrument 1030 via a smart electrosurgical connector 1020 (as discussed in FIG. 8). The second electrosurgical generator 1012 can control a surgical instrument 1032 via a smart electrosurgical connector 1022 (as discussed in FIG. 8). The third electrosurgical generator 1014 can control a surgical instrument 1034 via a smart electrosurgical connector 1024 (as discussed in FIG. 8).

The first smart electrosurgical connector 1020 can directly communicate 1015, 1025 with the second and third smart electrosurgical connectors 1022, 1024. Therefore, the smart electrosurgical connectors 1020, 1022, 1024 can share real-time information between them. Thus, an electrosurgical connector in a first room of a hospital can share data received from one or more surgical instruments in the first room with an electrosurgical connector in a second room of the hospital, which is connected to a plurality of other surgical instruments. Such data can be transmitted to a central database, where the data is analyzed.

The present invention may be a system, a method, and/or a computer program product at any possible technical detail level of integration. The computer program product may include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, configuration data for integrated circuitry, or either source code or object code written in any combination of one or more programming languages, including an object oriented programming language such as Smalltalk, C++, or the like, and procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

Reference in the specification to “one embodiment” or “an embodiment” of the present invention, as well as other variations thereof, means that a particular feature, structure, characteristic, and so forth described in connection with the embodiment is included in at least one embodiment of the present invention. Thus, the appearances of the phrase “in one embodiment” or “in an embodiment”, as well any other variations, appearing in various places throughout the specification are not necessarily all referring to the same embodiment.

It is to be appreciated that the use of any of the following “/”, “and/or”, and “at least one of”, for example, in the cases of “A/B”, “A and/or B” and “at least one of A and B”, is intended to encompass the selection of the first listed option (A) only, or the selection of the second listed option (B) only, or the selection of both options (A and B). As a further example, in the cases of “A, B, and/or C” and “at least one of A, B, and C”, such phrasing is intended to encompass the selection of the first listed option (A) only, or the selection of the second listed option (B) only, or the selection of the third listed option (C) only, or the selection of the first and the second listed options (A and B) only, or the selection of the first and third listed options (A and C) only, or the selection of the second and third listed options (B and C) only, or the selection of all three options (A and B and C). This may be extended, as readily apparent by one of ordinary skill in this and related arts, for as many items listed.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the blocks may occur out of the order noted in the Figures. For example, two blocks shown in succession may, in fact, be accomplished as one step, executed concurrently, substantially concurrently, in a partially or wholly temporally overlapping manner, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

Having described preferred embodiments of electrosurgical generators (which are intended to be illustrative and not limiting), it is noted that modifications and variations can be made by persons skilled in the art in light of the above teachings. It is therefore to be understood that changes may be made in the particular embodiments disclosed which are within the scope of the invention as outlined by the appended claims. Having thus described aspects of the invention, with the details and particularity required by the patent laws, what is claimed and desired protected by Letters Patent is set forth in the appended claims.

1. An electrosurgical generator for controlling a surgical instrument, the electrosurgical generator comprising:
a controller programmed to generate a first carrier wave signal and a second carrier wave signal based on an algorithm executed on a processor employing real-time current values of tissue at a tissue site to determine tissue impedance at a distal end of the surgical instrument, the first carrier wave signal having a first oscillating waveform and the second carrier wave signal having a second oscillating waveform;
a multistage variable gain amplifier for amplifying the first and second oscillating waveforms to generate a first output signal for a first mode of operation and a second output signal for a second mode of operation; and
an electrosurgical connector for transmitting the first and second output signals to one or more electrodes on the surgical instrument,
wherein the controller concurrently runs the first and second oscillating waveforms while switching between the first mode of operation and the second mode of operation.

2. The electrosurgical generator of claim 1, wherein a frequency of the first carrier wave signal and a frequency of the second carrier wave signal are modulated by employing the algorithm to optimize the frequencies of the first and second carrier wave signals based on the determined tissue impedance, phase angle, and reactance to tissue to maximize power output.

3. The electrosurgical generator of claim 2, wherein the frequency of the first carrier wave signal and the frequency of the second carrier wave signal are modulated in a frequency range from about 200 kHz to about 10 MHz.

4. The electrosurgical generator of claim 1, wherein the first oscillating waveform and the second oscillating waveform are formed within a rectangular modulation envelope, the first oscillating waveform forming first energy pulses of a first shape and the second oscillating waveform forming second energy pulses of a second shape.

5. The electrosurgical generator of claim 4, wherein the rectangular modulation envelope remains constant regardless of the first shape of the first energy pulse and the second shape of the second energy pulses formed therein.

6. The electrosurgical generator of claim 4, wherein the rectangular modulation envelope is not defined by the first shape of the first energy pulses and the second shape of the second energy pulses formed therein.

7. The electrosurgical generator of claim 1, wherein the first and second carrier wave signals are modulated with a digital pulse wave to create a repeating pulse wave cycle.

8. The electrosurgical generator of claim 1, wherein the first carrier wave signal and the second carrier wave signal are switched between an ON state and a LOW state to create high energy pulse waves and low energy pulse waves with ON times and OFF times between about 1 kHz and about 200 kHz, with a duty cycle of between about 1% to 99%.

9. The electrosurgical generator of claim 1, wherein the first carrier wave signal and the second carrier wave signal are switched between an ON state and an OFF state such that a switching frequency of ON times and OFF times is between about 1 kHz and about 200 kHz, with a duty cycle of between about 1% to 99%.

10. The electrosurgical generator of claim 1, wherein an inactive waveform signal is created when the first carrier wave signal and the second carrier wave signal are in an OFF state to allow communication between at least the controller, the multistage variable gain amplifier, an impedance matching module, and radio frequency identification (RFID) elements.

11. The electrosurgical generator of claim 10, wherein the first carrier wave signal and the second carrier wave signal are switched between ON times and OFF times at a frequency between about 1 Hz and about 1000 Hz, with an ON time duration between about 0.05 milliseconds to about 1000 milliseconds, and with an OFF time duration between about 0.001 milliseconds to about 10 milliseconds.

12. The electrosurgical generator of claim 1, wherein the first oscillating waveform is amplified independently of the second oscillating waveform.

13. The electrosurgical generator of claim 1, wherein pH levels of the tissue at the tissue site are measured to regulate output power.

14. The electrosurgical generator of claim 1, wherein, when pH levels of the tissue at the tissue site exceed one or more thresholds, the algorithm executed on the processor ends one or more power delivery modes.

15. The electrosurgical generator of claim 1, wherein a thermal camera is in communication with the electrosurgical generator to measure temperature gradients at the tissue site to regulate output power.

16. The electrosurgical generator of claim 1, wherein the electrosurgical connector includes a temperature sensor, a storage device, and a radio frequency identification (RFID) chip embedded therein, the RFID chip having an RFID antenna incorporated on an outer surface of the electrosurgical connector.

17. The electrosurgical generator of claim 1, wherein the electrosurgical connector includes an indicator on an external surface thereof to convey real-time information to a user.

18. The electrosurgical generator of claim 1, wherein the electrosurgical connector communicates directly with a plurality of other electrosurgical connectors coupled to a plurality of other surgical instruments.

19. The electrosurgical generator of claim 1, wherein the electrosurgical generator communicates with a central database over an Internet-of-Things (IoT) network, the communication including global positioning system (GPS) coordinates of the electrosurgical generator and/or the surgical instrument, and a timestamp.

20. The electrosurgical generator of claim 19, wherein the electrosurgical generator is calibrated remotely and firmware updated through the IoT network.

21. The electrosurgical generator of claim 1, wherein the electrosurgical generator is controlled by robotics.

22. The electrosurgical generator of claim 1, wherein the first and second oscillating waveforms are displayed on an augmented reality (AR) enabled display receiving information from an AR sensor positioned at the distal end of the surgical instrument.

23. The electrosurgical generator of claim 1, wherein a vacuum is created at the distal end of the surgical instrument to drop atmospheric pressure to reduce a boiling point of water in order to lower a temperature of ablation.

24. An electrosurgical generator for controlling a surgical instrument, the electrosurgical generator comprising:
a controller programmed to generate a carrier wave signal based on an algorithm executed on a processor employing real-time current values of tissue at a tissue site to measure at least one of phase angle and reactance of the tissue, the carrier wave signal having an oscillating waveform;
a multistage variable gain amplifier for amplifying the oscillating waveform to generate an output signal for selecting a mode of operation for the surgical instrument; and
an electrosurgical connector for transmitting the output signal to one or more electrodes on the surgical instrument,
wherein the controller modulates the frequency to reduce the at least one of the phase angle and the reactance of the tissue to maximize power output to the tissue.

25. The electrosurgical generator of claim 24, wherein the frequency is modulated from a range between about 200 kHz to about 10 MHz.

26. An electrosurgical generator for controlling a surgical instrument, the electrosurgical generator comprising:
a controller programmed to generate a first carrier wave signal and a second carrier wave signal based on an algorithm executed on a processor employing real-time current values of tissue at a tissue site to determine tissue impedance at a distal end of the surgical instrument, the first carrier wave signal having a first oscillating waveform and the second carrier wave signal having a second oscillating waveform;
a multistage variable gain amplifier for amplifying the first and second oscillating waveforms to generate a first output signal for a first mode of operation and a second output signal for a second mode of operation; and
an electrosurgical connector for transmitting the first and second output signals to one or more electrodes on the surgical instrument,
wherein the controller runs the first and second oscillating waveforms in an alternating manner while switching between the first mode of operation and the second mode of operation.

27. The electrosurgical generator of claim 26, wherein the first and second oscillating waveforms alternate at a frequency between about 0.1 Hz and about 200 kHz.
